(19) United States
(12) Patent Application Publication
(10) Pub. No.: US 2015/0192521 A1
(43) Pub. Date: Jul. 9, 2015

(54) METHOD, DEVICE AND PORTABLE METER FOR DETECTING DEGRADATION PRODUCTS OF BIOLOGICAL MOLECULES IN LAYERS OF A LAYER SYSTEM

(71) Applicant: Fraunhofer-Gesellschaft zur Foerderung der angewandten Forschung e. V., Munich (DE)

(72) Inventors: Holger Lausch, Jena (DE); Michael Brand, Jena (DE); Michael Arnold, Jena (DE); Joseph Maria Regina Delhaes, Muehlheim am Main (DE)

(21) Appl. No.: 14/409,116

(22) PCT Filed: Jun. 18, 2013
(86) PCT No.: PCT/DE2013/100220

§ 371 Date: Dec. 18, 2014

(30) Foreign Application Priority Data
Jun. 18, 2012 (DE) 10 2012 105 291.3

(51) Int. Cl. G01N 21/64; G01N 21/94

(52) U.S. Cl. CPC G01N 21/6486; G01N 21/94; G01N 2201/06113

(57) ABSTRACT

The invention is directed to a method for the detection of degradation products of biological molecules in layers of a layer system (1) in which the degradation products are acted upon by an excitation radiation (3) and a radiation (6) of the degradation products which is induced by the excitation radiation (3) is detected and a concentration of degradation products is determined based on a comparison of the measurement values of the radiation (6) with reference data, characterized in that there is provided a layer system (1) which includes a through layer (1.1) which is transparent to the excitation radiation (3) and radiation (6) and at least one further receiver layer containing degradation products, and in that the excitation radiation (3) is directed into the receiver layer through the through layer (1.1). The invention is further directed to an apparatus and a portable measuring device (11) for detecting the degradation products.

The invention is directed to a method for the detection of degradation products of biological molecules having certain characteristics through an excitation of the biological molecules by a radiation and the acquisition and evaluation of a radiation of the molecules which is remitted as a result of the excitation as is known generically from the document DE 103 15 541 A1. The invention is further directed to apparatus for the detection of biological molecules and to apparatus for carrying out the method.

Present-day high standards particularly in the domain of food hygiene at all stages of production, storage, sale and final consumption require methods by which unwanted or even harmful deviations from the standards to be complied with are detected in a simple yet dependable manner. In this regard, methods allowing a nondestructive and noncontacting detection of substances or groups of substances as indicators for a condition of a foodstuff or of another organic raw material or product or of raw materials or products containing organic components are particularly advantageous.

It is well known that there is a range of molecules which can be excited by applying a high-energy radiation of appropriate wavelength or wavelength spectrum for emitting radiation (autofluorescence).

In the following, emphasis will be on degradation products of biological molecules. Hereinafter, biological molecules are intermediate products or end products which have been synthesized by organisms. Degradation products are formed by metabolic processes or through degradations of chemical compounds, particularly biological molecules, brought about by organisms.

The basic procedure for utilizing autofluorescence for purposes of detecting degradation products of biological molecules is described, for example, in DE 103 15 541 A1. A method is described in which an energy flow from an energy source, e.g., a laser or a mercury vapor lamp, is directed to a surface of a foodstuff to be analyzed and fluorescable molecules are excited for emitting a fluorescent radiation. The intensity of the fluorescent radiation is detected as a function of its wavelength by means of a detector (e.g., spectrometer, photomultiplier) and compared with values from a reference library. The respective sample is analyzed in a nondestructive manner while avoiding removal of material. A concentration of (auto-)fluorescable molecules can be determined at least qualitatively by way of the detected values of the fluorescent radiation. The method is used primarily for process control and for timely removal of foodstuffs having measurement values indicating an insufficient degree of freshness. Also, a device is described which comprises at least one energy source, a detector for the emitted fluorescent radiation and an evaluating unit.

The above-stated principle has already been applied in an array of methods and devices, particularly for monitoring foodstuffs. For example, WO 90/01693 describes a method for visually inspecting organic materials by which impurities or foodstuffs contaminated with harmful organisms, e.g., the Aspergillus flavis mold which produces the toxin aflatoxin, can be detected and sorted out even on a large industrial scale. It is already known from EP 0 128 889 A1 to distinguish various tissue types and fractions thereof in meat of diverse provenance which is to be processed.

U.S. Pat. No. 5,474,910 A describes a possibility for detecting fluorescable biological molecules and living microorganisms. The description relates to a method and a handheld device for one-time or repeated comparative detection of fluorescing biological molecules or microorganisms in the form of specific spectral fluorescence “fingerprints” in a defined space or on a defined surface accompanied by irradiation with light of suitable wavelengths. The field of application ranges from detection of biological contaminants for military uses to in vitro or in vivo measurement of human and animal tissue surfaces.

The methods mentioned above have the disadvantage that the excitation of the biological molecules takes place at the surface of the foodstuff and the foodstuff must be unpackaged when examined. The prior art methods do not allow a detection of biological molecules in layers of a succession of layers such as is presented, for example, by a sequence of packaging film, a foodstuff and an intermediate space between packaging film and foodstuff.

A possibility of this kind is of particular interest, for example, when packaged foodstuffs are purchased by the end consumer and it is not possible to remove the foodstuff from its packaging before purchasing.

A variety of methods are known from the field of detection of specific molecules or molecule groups (e.g., US2005/0214789 A1, US 2001/0281775 A1, DE 699 11 062 T2) in which the molecules or groups of molecules to be detected (for example, having the same functional groups or the same or similar patterns of saturated and/or unsaturated bonds) can be detected by introducing chemical compounds or molecules as markers. These methods merely allow indirect determination of the chemical compounds or molecules to be detected. Either only the specifically bonded markers are detected or the molecules or molecule groups to be detected can only be detected through chemical alteration by introducing chemical compounds. In both cases, the molecules to be detected are changed from an original form to a detectable form. Procedures of this kind are impossible, e.g., in packaged foodstuffs because the foodstuff may not be removed from its packaging at least by a customer at the place of purchase (e.g., store, point of sale, POS), because the foodstuff is no longer saleable or even edible after such a procedure and because the customer cannot carry the elaborate analysis equipment and chemicals that would be required for detection of this type.

Therefore, it is the object of the invention to suggest a possibility for detecting degradation products of biological molecules in layers of a succession of layers. It is a further object of the invention to provide an apparatus for the detection of degradation products of biological molecules. A further object is to suggest a portable device for carrying out the method.

By means of a method for the detection of degradation products of biological molecules in layers of a layer system in which no chemical substances need be injected into the layer system to enable the detection of degradation products and in which the degradation products of biological molecules are acted upon by an excitation radiation and a radiation of the degradation products which is induced by the excitation radiation is detected and a concentration of degradation products is determined based on a comparison of the measurement values of the radiation with reference data, the above-stated object is met in that there is provided a layer system which includes a through layer which is transparent to the excitation radiation and the radiation and at least one further receiver layer containing degradation products, wherein the excitation radiation is directed into the receiver layer through the through layer. Degradation products are molecules which can occur during the decomposition of biological molecules. In this regard, the invention relates to molecules exhibiting autofluorescence. Degradation products can also be biological molecules (biologically active molecules) or biogenic compounds (molecules). The terms “degradation product(s)” and “degradation products of biological molecules” are used synonymously.

Preferably a radiation of the degradation products which is induced by the excitation radiation (hereinafter referred to simply as “radiation”) is detected. In this respect, it is not ruled out that a—usually smaller—portion of detected radiation proceeds from sources other than degradation products, for example, from impurities. In this case also, the measurement values of the radiation are compared with reference data. In case of very large components of detected radiation from other sources, an (“equivalent”) concentration of degradation products is determined through the comparison of the measurement values with the reference data. This concentration, while not reflecting the actual concentration, still gives an indication of the current state of the layer system just like the actual concentration. Accordingly, for example, when a permissible limit value is exceeded by a value of a theoretical concentration, this provides an indication that the state of the layer system lies outside of a permissible range, for example, is spoiled. The method according to the invention can be carried out in an advantageous manner without introducing into the layer system chemical substances which support, or even allow at all, a detection of degradation products. Accordingly, detection is possible without altering the makeup of the layer system, the chemical-material composition of the layer system or the specific (micro)climatic conditions of the layer system. Further, it is very advantageous that the layer system need not have an exactly defined composition or predetermined dimensioning. In relation to the example of packaged foodstuffs, this means that different quantities of pieces of meat can be present in a quantity of shells. The pieces of meat can have various thicknesses. Further, in addition to a film covering, non-woven inserts can optionally be present. The respective individual layer systems can be analyzed by means of one and the same method according to the invention. By chemical substances is meant here particularly molecules or elements which are suitable as markers and which are added so that molecules to be detected can be detected at all.

Further, the method according to the invention can be carried out without the layer system needing to be first arranged specifically for a detection as is required, for example, for the analysis of histological sections. Further, the method according to the invention can be carried out without altering the layer system, particularly without needing to take it apart completely or partially. As it relates to the example of detection of degradation products in packaged foodstuffs, this means that a packaging film need not be removed to carry out the method. Besides this, layers should also not be removed from the layer system in this way because this would alter the conditions for the further course of degradation so drastically (e.g., elimination of a protective atmosphere, access for germs from the environment, alteration of the microclimate in the layer system) that the method would only remain suitable for determining a current state but not for subsequent detections in the same layer system.

The method is also advantageous for successive detections of degradation products of biological molecules spaced over time (e.g., several hours or days) in one and the same layer system.

It is assumed in the following that the layer system is vertically oriented. Therefore, a first layer is located on top, while the further layers of the layer system are arranged successively beneath the first layer. When the layer system is oriented in space in another manner, the description is to be interpreted correspondingly.

The through layer is preferably the uppermost layer of the layer system. For example, it can be a plastic packaging film or a plastic cover such as commonly used for packaging foodstuffs, e.g., meats and sausages, fish, fruits or vegetables. The term “translucent” is also implicit in the term “transparent”.

A receiver layer is a layer of the layer system in which the presence of degradation products of biological molecules is investigated and a concentration of biological molecules that may possibly be present is to be determined. Therefore, a layer to be analyzed is also a receiver layer when it does not contain any degradation products. The examination of the relevant layer in accordance with the method is the only deciding factor.

The presence in the receiver layer of other molecules which are not to be considered as biological molecules or degradation products is not relevant to implementation of the method. The detected radiation is evaluated as a whole without regard to its origin.

The layer system is produced by a series of determined, directly successive layers. In a layer system of this kind, a receiver layer containing the degradation products can be a first film layer which can be present directly on the underside of the through layer, for example, as a thin layer (film) of a condensate. Under these two layers, there may be a gas layer, a second film layer on the surface of the foodstuff, the foodstuff itself and a bottom layer, e.g., a shell of plastic or cardboard. The bottom layer can be additionally surrounded underneath by a film, for example by edge areas of a film forming the through layer.

The method is preferably applied to a layer system having a through layer and a first film layer directly following the latter. The first film layer is preferably aqueous and can contain organic compounds, e.g., biogenic amines.

By the term “concentration” is meant hereinafter a determination of a specific concentration within the framework of error tolerances or merely a comparison with a previously determined limit value. In the latter case, it is only checked whether or not the limit value has been exceeded with sufficient probability. This type of configuration of the method according to the invention is sufficient for a binary decision such as whether or not to purchase the foodstuff. The determination of a specific concentration and the comparison with a limit value can be combined.

It is an important feature of the invention that the excitation radiation is directed to the receiver layer in such a way that the radiation brought about in the receiver layer by the excitation radiation can be detected. For this purpose, a beam path along which the excitation radiation is directed into the receiver layer to be examined and a beam path along which the radiation propagates in direction of a device for detecting the radiation, e.g., a camera, a spectroscope or a photomultiplier, are directed into the layer system in such a way that the two beam paths intersect in the receiver layer.

In a first embodiment of the method according to the invention, the excitation radiation is a light of known wavelength. The measurement values obtained through the radiation are evaluated colorimetrically.

A second embodiment of the method according to the invention is configured in such a way that the excitation radiation is suitable for exciting fluorescable biological molecules and fluorescable degradation products thereof, wherein the excited molecules emit fluorescent radiation. The fluorescent radiation of the excited molecules is the radiation to be detected.

By excitation radiation and the radiation proceeding from the excited molecules is also meant radiation spectra.

The fluorescable degradation products of biological molecules are preferably amines. For example, these amines can be primary amines, secondary amines or tertiary amines. In particular, the amines can be certain ptomaines, e.g., cadaverine (1,5-diaminopentane) or putrescine, which arise through the biochemical decomposition of proteins and amino acids. Therefore, amines and amine concentration are suitable as indicators for the biochemical condition of a foodstuff.

The excitation radiation preferably has at least one wavelength from a wavelength range of 250 to 400 nm.

In order to achieve a high precision and conclusiveness of the method according to the invention, the fluorescent radiation can be detected in only one wavelength (measurement wavelength) provided for detection. Fluorescent radiation is detected around this measurement wavelength only in a narrow wavelength range of, e.g., ±5 nm. This sharply reduces the detection of fluorescent radiation that does not originate from molecules to be detected or from other sources of fluorescent radiation.

Amines possess a —C—NH₂— group as functional group. By means of excitation of characteristic electronic quantum transitions in the region of the chemical bond of this functional group, a fluorescence (autofluorescence) is brought about at 485 nm. Therefore, it is advantageous for the reliability of the method according to the invention when the fluorescent radiation is detected in a narrow wavelength range around 485 nm, e.g., in a range from 475 to 485 nm.

In the configuration of the method, it is possible that the radiation induced by the excitation radiation is excited in the first film layer. During an enzymatic decarboxylation taking place, for example, during the decomposition inter alia of proteins and amino acids through microorganisms, carbon dioxide and water are also formed in addition to biogenic amines such as the aforementioned ptomaines for example. Therefore, decomposing organic substances, which also includes foodstuffs as understood here, always have an aqueous layer (e.g., a second film layer) at their surface. Further, a gas layer will form over its surface. The aqueous layer and the gas layer are layers of the layer system. The aqueous layer can be a second film layer (on the surface of the organic substance) and a third film layer (on the underside of the organic substance) which can contain organic compounds.

Formation of biogenic amines is prevented or retarded not only by a nitrogen-free or low-nitrogen packaging, e.g., of a foodstuff, but also by refrigeration thereof. Regardless of whether or not the cold chain has been guaranteed to be uninterrupted, the dew point of the humidity within the more or less tight packaging is reduced by refrigeration to the extent that the humidity generally condenses and possibly also crystallizes at the surface of the foodstuff (second film layer) and at the underside of the through layer (first film layer). Both the first film layer and the second film layer can contain organic compounds such as biogenic amines. As a result of the gas layer as transitory transitional layer, the distribution of any biogenic amines present in the first film layer as aqueous condensate layer is usually more homogeneous than in the second film layer. This is also true when no gas layer is present.

In a further embodiment, the method according to the invention can be characterized in that the concentration of degradation products is determined in a layer system in which at least the second film layer containing aqueous organic compounds is present, and a radiation of the degradation products of the second film layer is excited by means of the excitation radiation, and the radiation of the degradation products of the second film layer is detected. This radiation can be a fluorescent radiation. It can also be a radiation that can be evaluated colorimetrically, wherein a radiation of this type which can be evaluated colorimetrically can also contain wavelengths which can be evaluated by fluorescence spectroscopy.

The method can also be used to detect biological molecules and/or degradation products thereof in other layers of a layer system. When these layers are located directly beneath an outer layer of the layer system, this will be referred to hereinafter as a measurement in the near area (near-area measurement), whereas measurements at layers arranged farther away from an outer layer are referred to as far-area measurements (measurements in the far area).

To avoid interactions which are disadvantageous for the reliability of the method, it is advantageous when the excitation of the second film layer and the detection of the radiation of the biological molecules of the second film layer are carried out at different times than the excitation of the first film layer and the detection of the radiation of the degradation products of the first film layer.

Further, it is possible and advantageous that the concentration of degradation products is determined colorimetrically and by fluorescence spectroscopy at different times. In further embodiments of the method, excitations of different receiver layers and different options for evaluation can also be combined spatially and temporally in any manner.

A determined concentration can be compared with a permissible limit value. If it is determined that the limit value is exceeded with sufficient probability, an indication signal can be generated. The indication signal can be, for example, an optical or acoustic signal or can be realized as a combination of different types of signals.

In addition to a colorimetric or fluorescence-spectroscopic detection and determination of concentration of degradation products, an impedance of at least one of the layers of the layer system can be measured by impedance spectroscopy and the phase angle of the impedance can be determined. The amounts of impedances measured can also be determined.

It is further possible to measure impedances of different layers of the layer system and to determine a displacement, i.e., a difference, between the phase angles of the measured impedance. An impedance can be measured, for example, at the uppermost layer and at the bottommost layer of the layer system and the phase angle displacement and differences in amount can be determined. For this purpose, the layers analyzed by impedance spectroscopy need not be transparent.

It is possible in an advantageous manner to carry out a selection of the resonances typical of the molecules with respect to aggregate states of the analyzed layers by means of impedance spectroscopy. Accordingly, typical resonances for the degradation products can be selected. In so doing, no bond vibrations of the molecules are detected.

The electrodes needed to carry out impedance spectroscopy can be brought into contact from the outside at the through layer, the bottommost layer of the layer system or at both of these layers. In both cases, an impedance is measured near the through layer or bottommost layer (near-area measurement). A plurality of electrodes can be arranged at the same distance or different distances in each instance, wherein these electrodes can also be connectable to one another as needed in order to allow highly flexible measurements.

The concentration of degradation products determined on the basis of the radiation and the phase angle displacements and differences in amount determined by impedance spectroscopy measurements can be analyzed for congruence with one another. A congruence is present, for example, when the state of an analyzed foodstuff is found to be sufficiently similar in the different layers. On the other hand, if the findings in the different layers diverge from one another to an impermissible extent, no congruence is determined. For example, if the impedance-spectroscopic measurement at the bottommost layer of the layer system indicates a fresh foodstuff, while the fluorescence-spectroscopic measurement of a first film layer leads to the conclusion that the foodstuff is already several days old, repackaging or replacement of the non-woven insert (e.g., in case of packaged meat) could have been carried out.

The indication signal can be generated when no congruence is determined. Alternatively or in addition, an actuation signal could also be generated when congruence is determined.

It is possible to use the various measuring methods repeatedly. For example, either the concentrations or the impedances or the concentrations and impedances can be determined repeatedly over a period of time.

In a further development of the method according to the invention, at least one data connection to a virtual storage and computing unit is produced and steps of the method are carried out by means of the virtual storage and computing unit. The virtual storage and computing unit is preferably a dynamically variable unit which can also be adapted to an actual storage and computing requirement such as, for example, a “cloud” in a network such as the Internet.

The method according to the invention can also be supplemented in such a way that method-related data are conveyed to a reception device, for example, a database, of a recipient. Method-related data can be, for example, information about the place and time of a measurement, information about the layer system measured, and about the measurement values obtained when carrying out the method and the results of the evaluations. Further, method-related data can also contain information about a user, for example, the person carrying out the measurement. An identification of a measuring device used for carrying out the method can also be conveyed. A recipient can be a third party, for example, a regulatory authority in charge of monitoring compliance with regulations concerning foodstuff hygiene. A service provider, e.g., a consumer protection Internet portal, or the distributor or purveyor of the analyzed foodstuff can also be the recipient. This configuration of the method also makes it possible to exchange information with the recipient virtually in real time.

The method according to the invention makes it possible, for example, to inspect the condition of packaged foodstuffs at the point of sale. Further, it is possible to inspect the condition of transparently packaged foodstuffs repeatedly over a period of time. The method can also be integrated in storerooms or in refrigeration equipment by correspondingly configured devices so that it is possible to monitor the condition of the foodstuff occasionally or continuously. The inspection can include a qualitative detection of degradation products and/or biological molecules such as biogenic amines on the one hand and a quantitative determination of an intensity of a change of state, e.g., due to the determined concentration, on the other hand. While the upper side of a package for foodstuffs, as film, is usually transparent, the underside of the package can be opaque.

The above-stated object is further met through an apparatus for the detection of degradation products of biological molecules in layers of a layer system. The apparatus has an excitation source for providing and emitting an excitation radiation and a detection unit for detecting radiation that is brought about (induced) by the excited degradation products. In this regard, beam paths of the excitation source and of the detection unit are directed into a common measuring space. Further, a computing unit is provided for controlling the excitation source and for controlling the detection unit and for evaluating the detected radiation. The apparatus according to the invention is characterized in that the excitation source is arranged above a layer system located in the measuring space, wherein the layer system has at least one through layer which is transparent to the excitation radiation and to the radiation and at least a further layer after the through layer. Further, the beam path of the excitation source is directed at a radiation angle to the through layer in order to excite degradation products in at least one layer located below the through layer. The detection unit is arranged in such a way that a radiation of the degradation products which is induced by the excitation radiation can be detected by the detection unit.

In an advantageous configuration of the apparatus according to the invention, the detection unit is arranged perpendicularly over the layer system so that the beam path of the detection unit is directed perpendicular to the layer system. In so doing, it is advantageous that only the radiation angle need be changed in order to detect a radiation from different layers. In an advantageous manner, unwanted effects can also be minimized or blocked by an arrangement of this type. Accordingly, excitation and radiation induced by the latter in layers other than the layer to be analyzed can be ignored in a simple manner because the beam path of the detection unit is not directed into the region of the other layer or layers in which the excitations occurring as secondary effects take place. The beam paths of the excitation unit and of the detection unit preferably intersect in that layer in which the excitation of the degradation products takes place.

In further embodiments of the apparatus according to the invention, the radiating angle can also be identical, but the excitation sources can then be arranged at different distances from the detection unit and from the beam path thereof, respectively, so that the beam paths intersect in the layer to be analyzed.

Further configurations of the apparatus according to the invention can have an excitation source formed of a plurality of controllable excitation units each having its own beam paths. Further, the excitation units can emit an excitation radiation with individual wavelength in each instance and can have beam angles which differ from one another.

Further, electrodes can be provided for measuring impedances. These electrodes can be arranged in pairs or as individual electrodes. The electrodes can be actuated individually and can be interconnected electively.

In order to make the method according to the invention actually also available for use by an end consumer, the above-stated object is further met by a portable measuring device for mobile detection by means of the method according to the invention. The measuring device has a receiving shell for receiving a telecommunications device. A receiving shell within the meaning of the description can be shaped similar to a holder for mobile phones, for example for use in motor vehicles (hands-free sets). A telecommunications device can be, for example, a mobile phone, a computer, a device suitable for home electronics, or a measuring device. Future technological developments in the field of telecommunications devices are hereby incorporated in the description.

An adapter by means of which a power supply of the measuring device is produced through the power source of the telecommunications device is preferably arranged in the receiving shell. Further, it is possible to control the measuring device via the adapter in that a program is installed in the telecommunications device which can be operated by means of the telecommunications device. This can be realized by means of correspondingly arranged and configured contacts of the adapter.

The data connection between the portable measuring device and the virtual storage and computing unit can be produced by the telecommunications device.

The invention is particularly suitable for a simple, noncontacting and nondestructive inspection of foodstuffs. The method according to the invention can also be used when one of the film layers is frozen.

In practice, a typically packaged foodstuff is not impervious to possible tampering by which a formation of degradation products such as biogenic amines and/or an interruption in the legally prescribed cold chain could be disguised chemically, visually or by means of relabeling and repackaging. Whereas foodstuff inspection authorities may remove packaging from foodstuffs at any time during a random sampling for inspection purposes, this is generally impossible for the customer at the point of sale. The invention proposes a method and options for applying the method which enable a customer to make purchasing decisions based on objective measurement data. The customer can also track the condition of purchased goods over a period of time in order to make a decision, e.g., about when to consume the foodstuff or goods.

According to the invention, this is achieved via a movable hand-held device for nondestructive and noncontacting qualitative detection and quantitative determination of the intensity of biogenic substance groups by means of which a dual sensor-actuator measuring method can be applied. In contrast to the monitoring variants in customers' homes, the quantitative and qualitative determination at point of sale cannot be carried out in an alternating manner over the course of time. As a result, the customer has only one opportunity for inspection and the inspection must be carried out in different layer systems and with unknown through layers. The invention enables inspection (detection and concentration determination) regardless of the properties of the through layer.

If the customer has the foodstuff within his sphere of influence, e.g., at home, more than one inspection is possible. It is also possible for the customer to preserve the foodstuff in a container, for example. Properties of the lid of the container representing the through layer can be known through reference measurements and be taken into account in subsequent inspections.

Some additional remarks should be made with regard to the chronology of the chemical and biochemical processes as foodstuffs age and spoil. While these processes are slowed down by a continuous cold chain, they can generally not be halted. Whereas NADH (nicotinamide adenine dinucleotide) occurs in meat and can be detected by conspicuous fluorescence after three days, fluorescence which is detectable by means of PCA (protein-fragment complementation assay) as well as changes in smell which can be clearly perceived by the olfactory senses occur in unpackaged meat after 7 to 8 days. However, the fluorescence is only detectable by weak Raman effects. After 10 to 11 days, there is a significant rise in the concentration of amines that are detectable by means of fluorescence. After 13 days, the microbiological bacterial count limit is reached, and after 16 days meat is deemed to be spoiled, which can be detected by laser-induced Raman fluorescence. After 20 days, the meat is infested with microbes and NADH of the microorganisms is detectable by means of fluorescence. Concurrently, there are also significant cyclical changes in pH, conductivity, impedance and temperature. The first and last of these changes are virtually undetectable in the case of refrigerated, packaged foodstuffs without direct contact or sampling. Fast physical measurements of conductivity and impedance are therefore additional indicators for the respective quality status and should therefore be included by way of supplementing quality determination with spectral-optical methods. For example, the phase angle displacement in impedance measurement exhibits complementary effects compared with the change in fluorescence intensity such as the zero-crossing and renewed rise in phase angle displacement when bioamines develop and increase (see the presentation by Schwägele, http://download.messemuenchen.de/analytica/Presentation/BM_Presentation_{—}020408/Sch waegele.pdf).

The method according to the invention, the apparatus according to the invention and the portable measuring device according to the invention can also be used for detection of biological molecules in other layer systems. For example, a detection and concentration determination of glucose or other hydrocarbons in human and animal epidermis are also possible.

The invention is described more fully in the following with reference to embodiment examples and drawings. The drawings show:

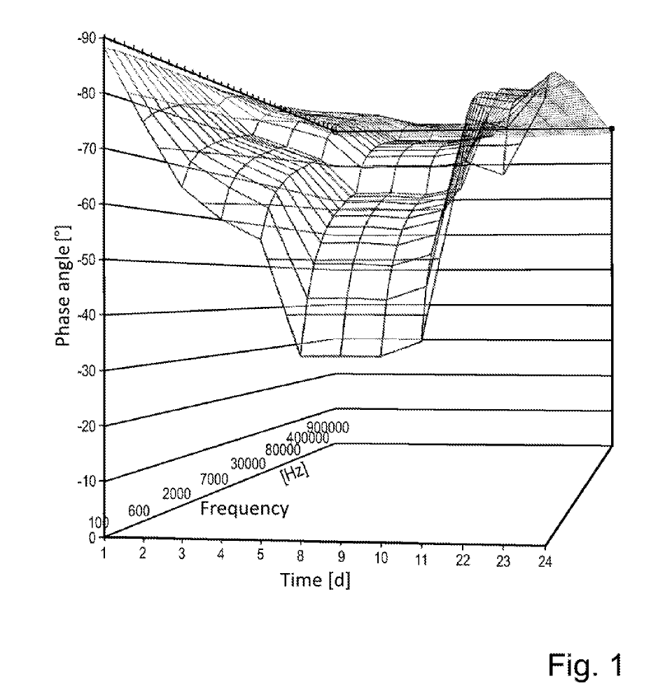

FIG. 1 the frequency-dependent change in the phase angle in measurements performed on pork by impedance spectroscopy over a time period of 24 days;

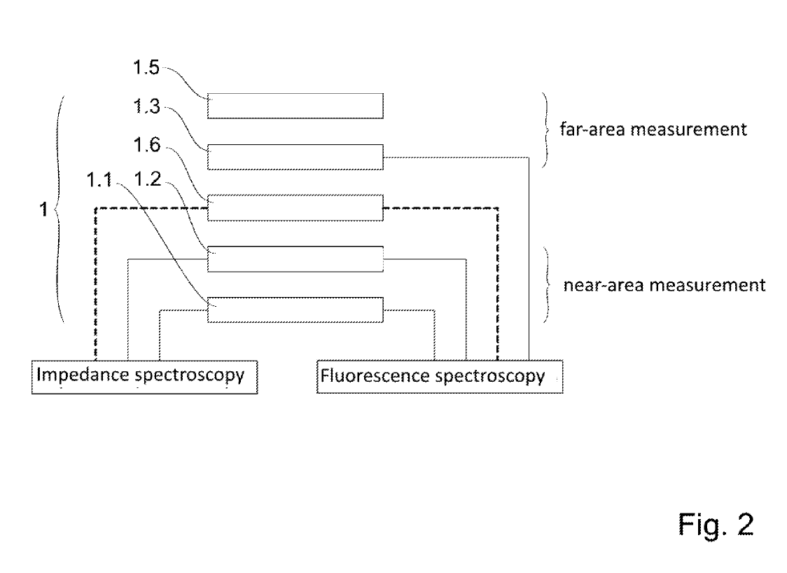

FIG. 2 a highly schematic diagram showing a first layer system and various procedures of detection of degradation products of biological molecules associated with the layers of the layer system;

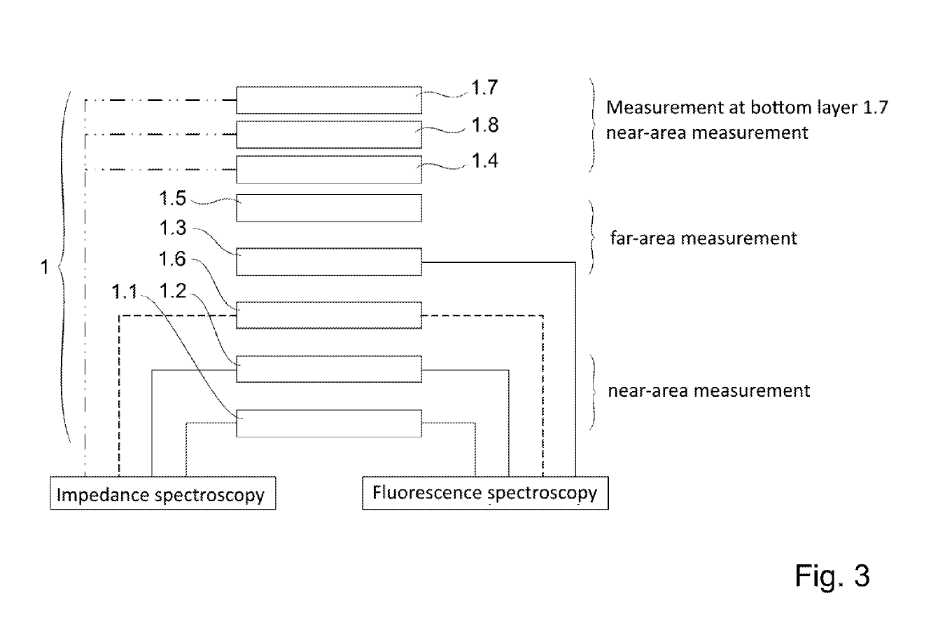

FIG. 3 a highly schematic diagram showing a second layer system and various procedures of detection of degradation products of biological molecules associated with the layers of the layer system;

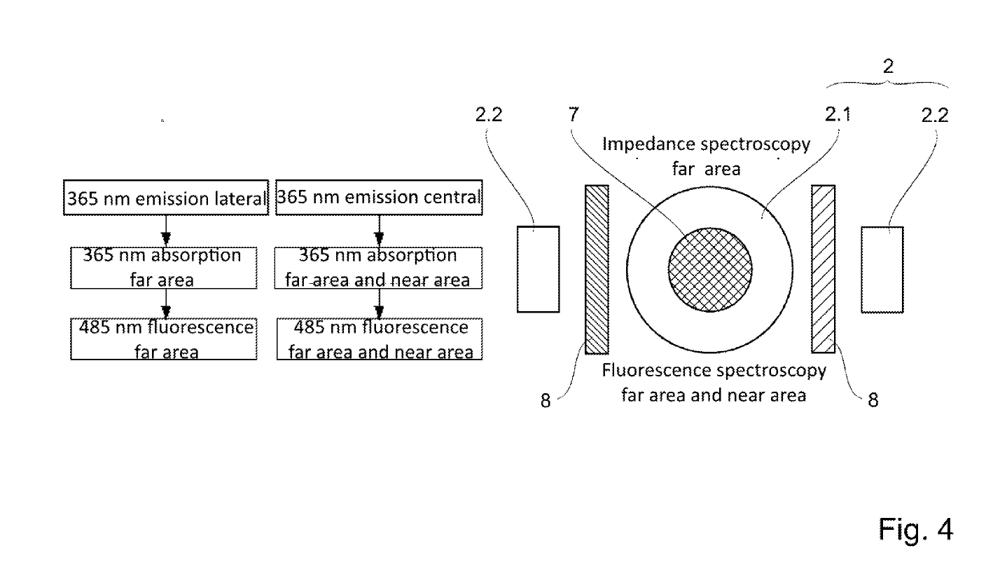

FIG. 4 a bottom view of a first embodiment example of the apparatus according to the invention with a first arrangement of excitation sources, electrodes and a detection unit;

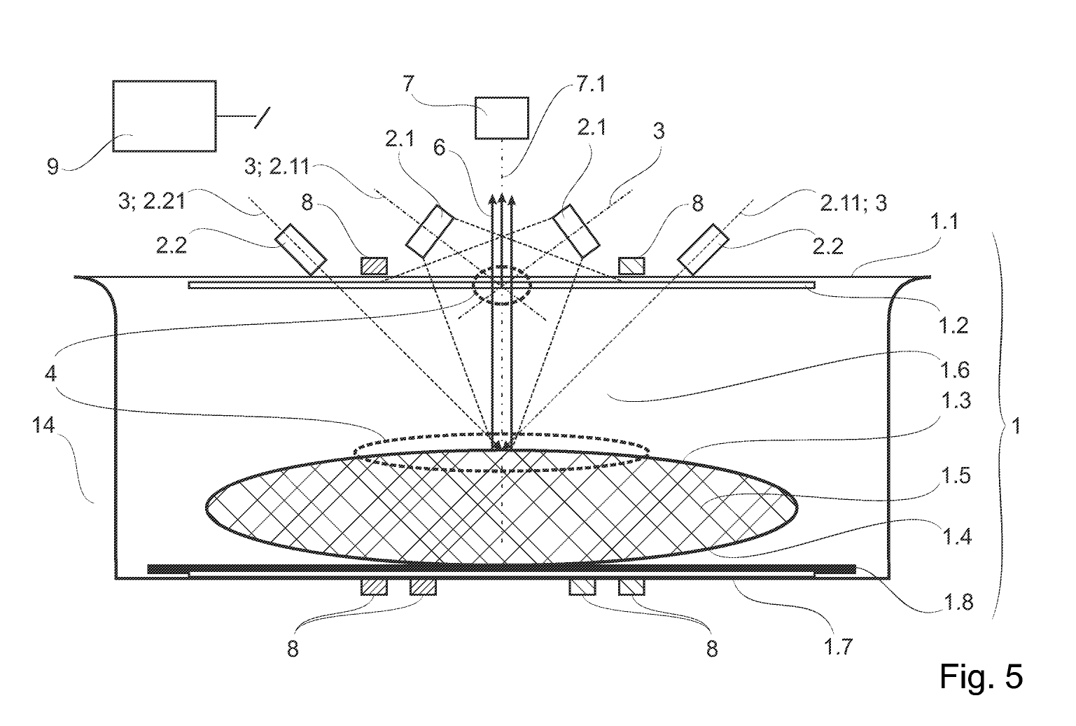

FIG. 5 a side view of the first embodiment example over a layer system;

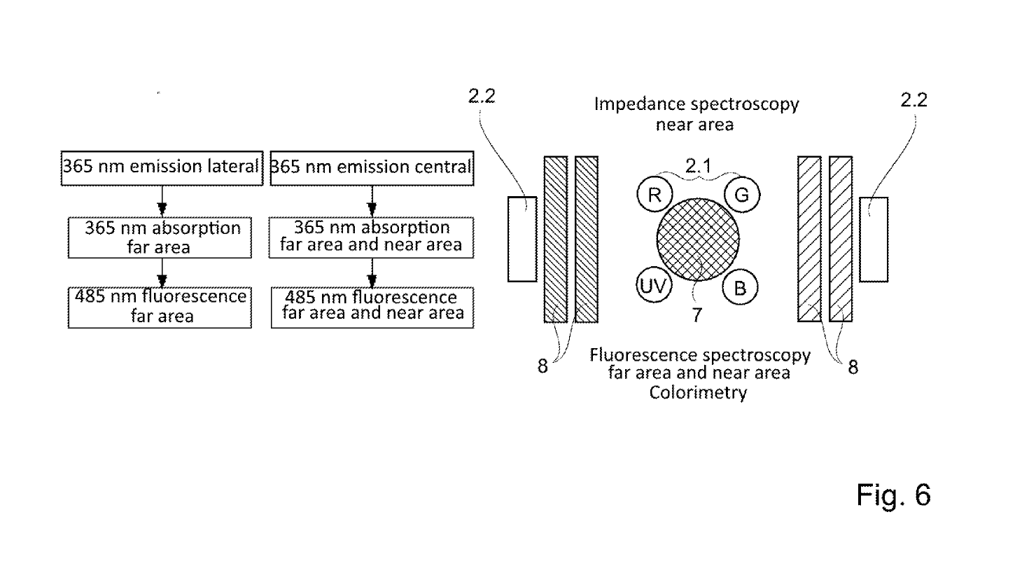

FIG. 6 a bottom view of a second embodiment example of the apparatus according to the invention with a second arrangement of excitation sources, electrodes and a detection unit;

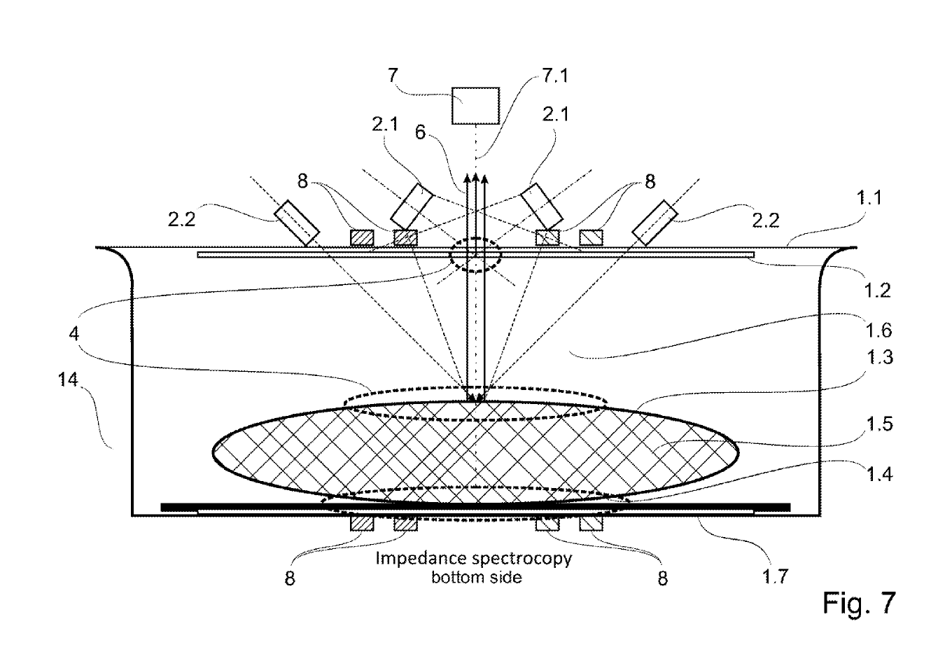

FIG. 7 a side view of the second embodiment example over a layer system;

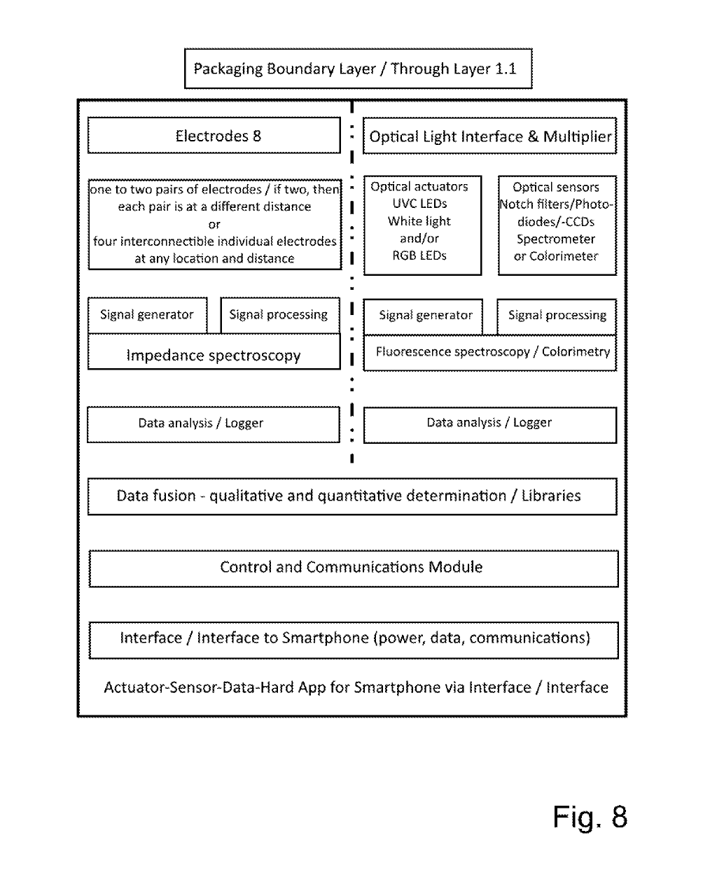

FIG. 8 a schematic diagram showing the detection of degradation products of biological molecules by means of impedance spectroscopy and excitation-based methods of fluorescence spectroscopy and colorimetry;

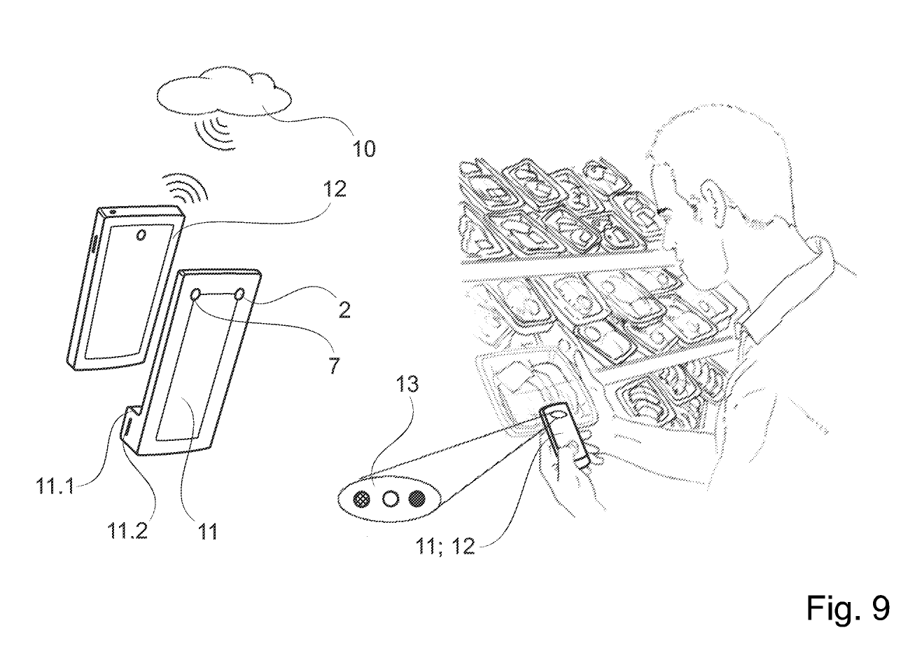

FIG. 9 a first embodiment example of a portable measuring device and use thereof;

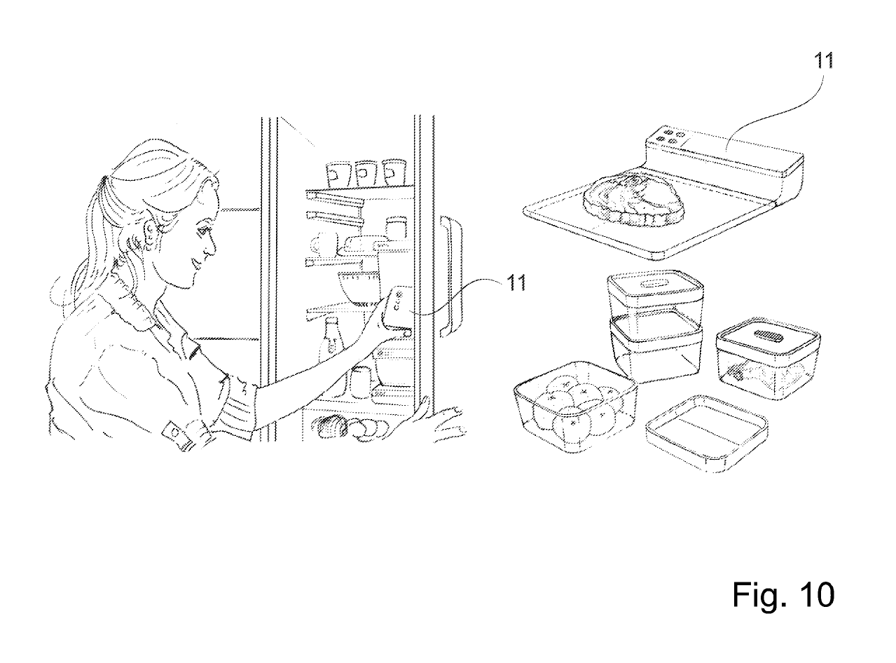

FIG. 10 a second embodiment example of a portable measuring device and use thereof;

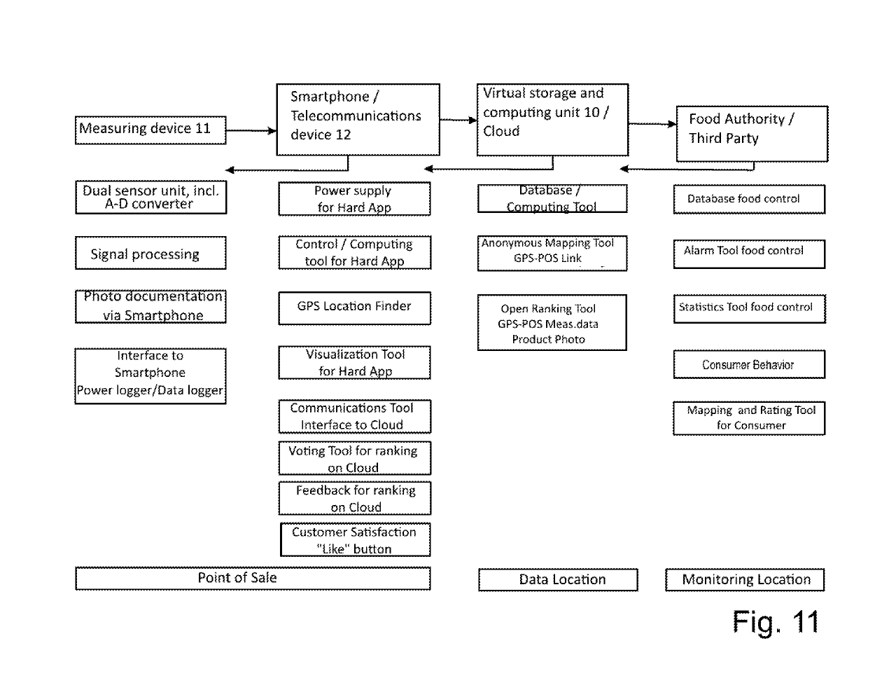

FIG. 11 a schematic overview of communications paths between a portable measuring device according to the invention, a virtual storage and computing unit and a reception device and functions and components of the aforementioned components;

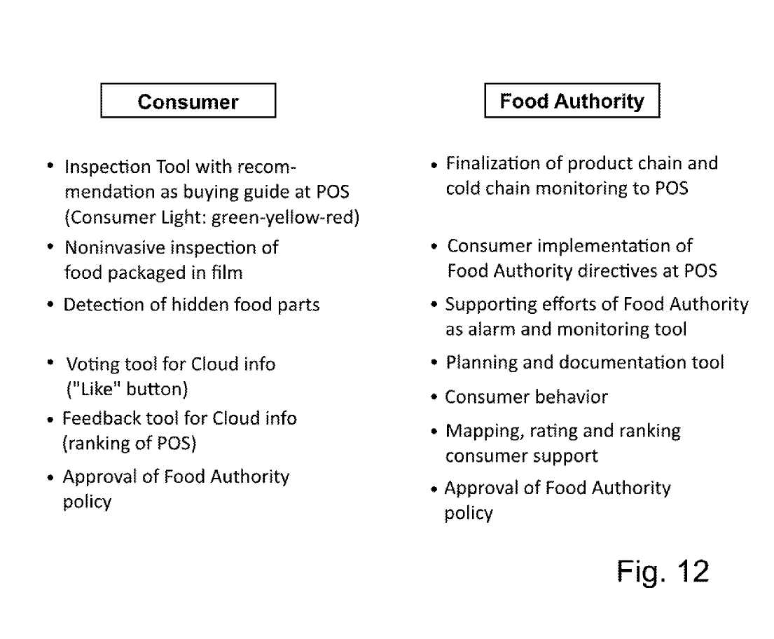

FIG. 12 an overview diagram of advantages of the invention for a consumer and a third party.

FIG. 1 shows a decrease in the amount of a phase angle φ determined by impedance spectroscopy over the first eight days of storage of pork. This can be attributed inter alia to a decrease in NADH on the meat. Between the eighth and tenth day, the phase angle φ is approximately constant, which can be explained by an equilibrium situation in the decomposition of NADH of the meat and the increase in NADH through a microbial colonization of the meat. After about the eleventh day, the phase angle φ rises again significantly through an increase in the concentration of microorganisms and metabolic activities thereof.

FIG. 2 shows a first layer system 1 with five layers which are listed from bottom to top for purposes of graphic depiction. The top layer of the depicted layer system 1 is a film as through layer 1.1 which is transparent to a utilized excitation radiation 3 (see FIG. 5) and to a radiation 6 of the degradation products of biological molecules (see also FIG. 5) which is caused by the excitation radiation 3. Under this top layer, there follows a first film layer 1.2 which is formed by aqueous condensation at the underside of the through layer 1.1. There follows a gas layer 1.6, a second film layer 1.3 and a foodstuff 1.5. The measuring processes of impedance spectroscopy and fluorescence spectroscopy and their applicability for measurement in the various layers of the layer system 1 are shown schematically. Measurements at the through layer 1.1 and first film layer 1.1 are possible as near-area measurements; measurements at the second film layer 1.3 and at the foodstuff 1.5 are carried out as far-area measurements. The gas layer 1.6 is not measured directly by any of the indicated methods but rather forms the boundary between near-area measurements and far-area measurements. However, the gas layer 1.6 exerts an influence on the corresponding methods which is symbolized by the dashed line.

FIG. 3 shows a layer system 1 having under the foodstuff 1.5 a third film layer 1.4, an absorbent pad (e.g., non-woven insert) as absorbent layer 1.8 and a nontransparent shell of plastic as bottom layer 1.7. If electrodes 8 (see FIG. 5) are arranged at the bottom layer 1.7, measurements can be carried out at the bottom layer 1.7, absorbent layer 1.8 and third film layer 1.4 by impedance spectroscopy. In further embodiments, the absorbent layer 1.8 can also be omitted.

A first arrangement of excitation sources 2, electrodes 8 and a detection unit 7 is shown in FIG. 4. The detection unit 7 is surrounded annularly by a UV source as first excitation source 2.1. Two further, separately arranged, second excitation sources 2.2 are arranged each on one side of the detection unit 7 and the first excitation source 2.1. An electrode 8 is provided in each instance between the first excitation source 2.1 and the second excitation sources 2.2. The first excitation source 2.1 and second excitation sources 2.2 emit radiation with a wavelength of 365 nm. The detection unit 7 is designed for detection of electromagnetic waves with a wavelength in a range of 485±5 nm.

The second excitation sources 2.2 are provided for an excitation of biological molecules in layers of a layer system 1 (see FIG. 5), while the first excitation source 2.1 is intended to cause excitations in the near area.

FIG. 5 presents a side view of the arrangement from FIG. 4 and additionally shows a layer system 1 in a measuring space 14, which layer system 1 comprises a through layer 1.1, a first film layer 1.2, a gas layer 1.6, a second film layer 1.3, a foodstuff 1.5, a third film layer 1.4, an absorbent layer 1.8 and a bottom layer 1.7 as well as two additional electrodes 8 on the through layer 1.1. The first excitation source 2.1 is shown in section. An intensity and distribution of the excitation radiation of the first excitation source 2.1 is selected such that an excitation region 4 is brought about in the first film layer 1.2 in which the excitation radiation 3 of the first excitation source 2.1 is sufficient for exciting biological molecules present in the first film layer 1.2 for emission of a fluorescent radiation as radiation 6. The detection unit 7 is arranged perpendicularly above the layer system 1 and a beam path 7.1 of the detection unit 7 is directed perpendicular to the layer system 1. The beam path 7.1 runs through the excitation region 4, where it intersects the beam paths 2.11 of the first excitation source 2.1. Measurements in the near area are made possible by the first excitation source 2.1.

The beam paths 2.21 of the second excitation sources 2.2 are directed to the second film layer 1.3 and intersect the beam path 7.1 of the detection unit 7 in the second film layer 1.3. A far-area measurement of the second film layer 1.3 is made possible by the second excitation sources 2.2. Four electrodes 8 are arranged in each instance on the outer sides of the through layer 1.1 and bottom layer 1.7. These electrodes on each of the layers can be connected to one another electively such that impedance-spectroscopic measurements are possible in a flexible manner.

To analyze impedance, pairs of electrodes 8 are positioned closer to one another and farther apart from one another on top of the layer system 1. These electrodes 8 are used for near-area measurement of the layer system 1. The first film layer 1.2 at the underside of the through layer 1.1 can be strengthened, or produced at all, simply by pressing on the through layer 1.1 until contacting the foodstuff 1.5 or by shaking or rotating the packaged foodstuff 1.5. The apparatus is controlled by means of a computing unit 9.

FIG. 5 shows the embodiment example in which the second film layer 1.3 and the upper surface of the foodstuff 1.5 are briefly irradiated by the second excitation sources 2.2 having a wide beam angle which are located further outward and are formed of LEDs with UV light to generate fluorescence at the upper surface of the foodstuff 1.5 which is detectable via the detection unit 7. Since the first film layer 1.2 is not irradiated directly below the detection unit 7, only the far-area measurement of a fluorescence at the second film layer 1.3 is detected. If only the first excitation source 2.1 which is located farther inward and which is likewise realized as an LED UV source is activated, only the first film layer 1.2 below the detection unit 7 and, to a lesser extent, the more remote layers are irradiated. Accordingly, the near-area measurement of fluorescence can be carried out. Additionally, both near-area measurements and far-area measurements can be measured in parallel as mutually strengthening or mutually overlapping when all of the excitation sources 2 are activated. Temporally successive measurements are also possible. Impedance spectroscopy can be carried out so as to complement the fluorescence spectroscopy.

In a second embodiment example shown in FIG. 6, the first excitation source 2.1 is formed of a UV source, a red light source, a blue light source and a green light source.

As can be seen in FIG. 7, excitation regions 4 are formed in the first film layer 1.2 and second film layer 1.3. An area in which an impedance-spectroscopic near-area measurement can be carried out by the electrodes 8 at the bottom layer 1.7 is highlighted in the third film layer 1.4.

The so-called triple mode allows a differential assessment of the homogeneity and intensity of fluorescing degradation products and therefore provides data on the current condition of the foodstuff 1.5. This also includes the complementary impedance-spectroscopic bottom side (measurement in the near area at the bottom layer) effect. If there is no typical congruence shown by the near-area measurements at the through layer 1.1 and bottom-side measurement of phase shift and near-area and far-area measurement of fluorescence, this indicates that the foodstuff 1.5 has been repackaged, cleaned or chemically treated or that the labeling has been altered. In case of unfrozen foodstuffs 1.5, the printed expiration date or consume-before date may not exceed 16 days back. This date can also be verified by reverse calculation and measurement. If the near area and far area coincide to some extent because of the type of packaging with the gas layer 1.6 largely absent, the second excitation sources 2.2 can be switched off. The fluorescence is detected and measured in the detector unit 7.

Optical light interfaces in the apparatus connect to a photodetector in the form of photodiodes, CCDs, spectrometers or colorimeters directly or via light guides and, where appropriate, via photomultipliers and notch filters.

For safety reasons and to prevent damage to health by exposure to high-power UV radiation, it is advantageous when the UV emission is linked to the placement of the electrodes 8 for impedance spectroscopy at the layer system 1.

Every chemical substance absorbs electromagnetic waves (light). In doing so, the energy of the light is used to execute certain movements of the respective substance-specific molecule (first case) or to excite electronic energy levels of the bonds and the atoms of these molecules (second case). In the first case, the energy is converted into kinetic energy. In the second case, electrons are raised to higher energy levels. When falling back to the low initial energy levels, the radiated energy is given again as electromagnetic wave. The energy (frequency, wavelength or wave number) of the emitted electromagnetic wave is the same as the coupled-in electromagnetic wave.

The shorter the wavelength and the higher the frequency, the greater the energy of an electromagnetic wave. This is described by the Einstein/de Broglie equations.

Through coupling of the second case with various processes of the first case, the molecule undergoes radiationless loss of energy. The light which is emitted again now has less energy than the injected light. Fluorescence is an effect of this type, for example.

The frequency of the fluorescent light is associated precisely with a defined quantum-mechanical electronic energy level transition and is therefore substance-specific or specific for a group of substances in a first approximation. The frequency of the excitation light must be higher than the frequency of the fluorescent light.

When a ptomaine-type chemical compound is irradiated with UV light, many ptomaines exhibit in a first approximation a characteristic fluorescence with a wavelength of 485 nm. The visible light comprises the wavelength range from 700 to 400 nm.

The wavelength of the radiated UV light can be 380 nm, 365 nm, 320 nm or 254 nm. The substances will fluoresce in every case. However, there is an optimum at which all other effects induced by an electromagnetic wave are minor.

Ptomaines are inter alia amines such as cadaverine (1,5-diaminopentane) which is formed through biochemical decomposition of proteins and amino acids inter alia through decarboxylation. The functional amino group ( . . . —C—NH₂) is characteristic of this group of substances. The fluorescence at 485 nm occurs through the excitation of characteristic electronic quantum transitions in the area of the chemical bond of this functional group.

According to FIGS. 6 and 7, UV light with a wavelength of 365 nm is generated by means of a plurality of UV diodes (UV LEDs). The UV light is guided centrically to the through layer 1.1 and the first film layer 1.2 and acentrically to the foodstuff 1.5. The excitation radiation 3 that is guided in a centric annular manner traverses the through layer 1.1 and the first film layer 1.2. In so doing, fluorescence is excited in the ptomaines in the condensate of the first film layer 1.2 (near area). The fluorescent light is guided as radiation 6 through a coaxial light guide (in the interior of the annularly guided first excitation source 2.1) to the detection unit 7 and is evaluated. The input of the light guide “looks” in the direction of the centrically guided light. The output terminates in the detection unit 7.

The acentric light reaches the foodstuff 1.5 and second film layer 1.3, where it excites fluorescence in the ptomaines which are present therein. When focusing, the fluorescent light can also be “seen” by the aforementioned coaxial light guide (far area). Without the methods of focusing, a plurality of excitation UV LEDs can also be used as first excitation source 2.1 and/or second excitation sources 2.2 while generating broader light spots.

The detection unit 7 evaluates only a certain very narrow wavelength range (485±5 nm) of the light. Extraneous visible light (radiation 6) and the exciting UV light as excitation radiation 3 are blocked by cut-off filters.

In FIGS. 6 and 7, the excitation of fluorescence is carried out as described above. Additionally, however, white light and/or RGB LEDs (red-green-blue semiconductor light emitting diodes) are provided in the near area as first excitation sources 2.1 and as a spectroscopic and/or colorimetric unit as detection unit 7. Aside from the respective resources with respect to space, cost and data, the distinctive characteristic of colorimetry as alternative and/or supplement to conventional fluorescence spectroscopy by means of spectrometers or wave range-optimized photodiodes with notch filters is the recalculation of the spectral measurement values of the colorimeter to the desired color coordinates.

An extensive computation which is inherent to the method can be outsourced to external servers. The tristimulus values defined by the International Commission on Illumination (CIE) have been generally adopted. These base numbers are available in tabulated form at a distance of one nanometer. However, the radiation proceeding from the light source is changed by the spectral characteristics of the relevant surface in case of object colors or transmitted colors. For the color stimulus function impinging on the eye, the color in the actual sense, this “affected” spectral radiance must be used. This is either the spectral remission curve in surface colors or the spectral transmittance curve in transmitted colors. During evaluation, the spectral measurement value is determined using a set of tabulated standard values at suitable reference points. In this case, the selected coefficients for remission and transmission are determined and added. Considered the other way around, the radiation distribution of the light source can also be taken together and measured in this spectral interval. Correspondingly, the color values result from measurement of the color stimulus in these intervals.

In the case of application, the remission of the light that is radiated in is likewise changed by the surface of the layers. When fluorescence effects are added, the spectral components under the fluorescent color are absorbed. In the least favorable case, with a blue fluorescence, “blue” from the RGB triplet is weakened through absorption. A spectral line with a color stimulus is remitted, which spectral line is located in the additive region of green and blue. This alters the histogram of the RGB triplet over the surface. There results an altered color stimulus which is evaluated as described above.

The absorption of “blue” described above is inconsequential, as the RGB signal is radiated in the entire measurement interval. This becomes clear when considering a white surface which fluoresces blue. Therefore, a violet color impression is not produced.

Since the foodstuffs have different natural or artificial colorations and the background color of the packaged foodstuff (bottom layer 1.7) can vary from light to jet black, the arrangement shown in FIGS. 6 and 7 is advantageous for colorimetry.

A schematic overview of the individual method steps and of some elements of the apparatus is shown in FIG. 8.

A portable measuring device 11 for carrying out the method is shown schematically in FIG. 9. The measuring device 11 contains an excitation source 2 and the detection unit 7. Further, a receiving shell 11.1 which serves to receive a telecommunications device 12 is formed integral therewith. A connection to the power supply and to the data link of measuring device 11 and telecommunications device 12 is realized via an adapter 11.2 of the measuring device 11. The measuring device 11 can be operated by means of the telecommunications device 12 via a program installed in the measuring device 11. Further, communication with a virtual storage and computing unit 10 which provides a part of the computing resources and the storage capacity required for implementing the method according to the invention is made possible via the telecommunications device 12. Further, the communication with the virtual storage and computing unit 10 serves for communication with a reception device (not shown), e.g., a database which can be updated online or a third-party electronic mailbox system. This communication can also be carried out through the telecommunications device 12 via the customary telecommunications channels. The use of the measuring device 11 with connected telecommunications device 12 by a user at a point of sale of packaged foodstuffs is shown on the right-hand side of the drawing.

The user presses the measuring device 11 connected to the telecommunications device 12 on a layer system 1 to be analyzed, in this case a piece of meat which is packaged in film, and actuates the measuring device 11 via the telecommunications device 12. Depending on the specific configuration of the measuring device 11, at least one fluorescence-spectroscopic and/or colorimetric measurement of the first film layer 1.2 of the layer system 1 is carried out. The acquired radiation 6 of the excited degradation products is evaluated, a concentration of the degradation products is determined and is compared with a permissible limit value. A display 13 which indicates via a color code whether or not the limit value has been exceeded by green and red areas of the display 13 is displayed on the telecommunications device 12. During the measurement or when the measurement results are ambiguous, a yellow area is displayed. In further embodiments of the measuring device 11, a signal tone can be emitted. In order to monitor the measurement, the user can carry out an impedance-spectroscopic measurement of the bottom layer 1.7 so that the measuring device 11 can analyze the measurements of both methods for congruence with one another. The user is alerted when there is an impermissible deviation. At the same time, information can be sent to the responsible food inspection authorities.

A stationary embodiment of the apparatus according to the invention is shown in FIG. 10. An apparatus according to the invention can be integrated in a refrigeration device or can be provided as a household appliance. In further embodiments, the apparatus according to the invention can also be designed for inspection and monitoring purposes of consumer protection agencies, for example, or with authorities responsible for monitoring foodstuffs and can be provided with a corresponding inspection certification, for example.

FIG. 11 shows possible sub-steps of the method and options for linking these sub-steps.

Options for interactions and data usage between a user and a third party such as food monitoring authorities are shown in an overview in FIG. 12.

### LIST OF REFERENCE NUMERALS

1 layer system
1.1 through layer
1.2 first film layer
1.3 second film layer
1.4 third film layer
1.5 foodstuff
1.6 gas layer
1.7 bottom layer
1.8 absorbent layer
2 excitation source
2.1 first excitation source
2.11 beam path (of the first excitation source 2.1)
2.2 second excitation source
2.21 beam path (of the second excitation source 2.2)
3 excitation radiation
4 excitation region
5 radiating angle
6 radiation
7 detection unit
7.1 beam path (of the detection unit 7)
8 electrodes
9 computing unit
10 virtual storage and computing unit
11 measuring device
11.1 receiving shell
11.2 adapter
12 telecommunications device
13 display
14 measuring space
φ phase angle

1.-25. (canceled)

26. A method for the detection of degradation products of biological molecules in layers of a layer system, wherein no chemical substances are introduced into the layer system to enable the detection of degradation products, wherein the degradation products are acted upon by an excitation radiation and a radiation of the degradation products which is induced by the excitation radiation is detected and a concentration of degradation products is determined based on a comparison of measurement values of the radiation of the degradation products with reference data, and wherein there is provided a layer system which comprises a through layer which is transparent to the excitation radiation and the radiation of the degradation products and at least one further receiver layer containing degradation products, the excitation radiation being directed into the receiver layer through the through layer.

27. The method of claim 26, wherein the excitation radiation is a light of known wavelength, and the measurement values are evaluated colorimetrically.

28. The method of claim 26, wherein the excitation radiation serves to excite fluorescable degradation products of biological molecules for emission of fluorescent radiation, and the radiation of the degradation products is a fluorescent radiation of the excited molecules.

29. The method of claim 28, wherein the degradation products are fluorescable amines.

30. The method of claim 28, wherein the excitation radiation has at least one wavelength from a wavelength range of 250 to 400 nm.

31. The method of claim 30, wherein the fluorescent radiation is detected in a wavelength range of ±5 nm around a wavelength provided for detection of fluorescent radiation.

32. The method of claim 26, wherein the radiation of the degradation products is excited in a first film layer of the layer system.

33. The method of claim 26, wherein the concentration of the degradation products is determined in a layer system in which at least one second film layer containing aqueous organic compounds is present, and an emission of the radiation of the degradation products of the second film layer is excited by the excitation radiation, the radiation of the degradation products of the second film layer being detected.

34. The method of claim 33, wherein excitation of the second film layer and detection of the radiation of the degradation products of the second film layer are carried out at different times than an excitation of a first film layer and a detection of the radiation of the degradation products of the first film layer.

35. The method of claim 27, wherein the concentration of degradation products is determined colorimetrically and by fluorescence spectroscopy at different times.

36. The method of claim 26, wherein a determined concentration is compared with a permissible limit value and an indication signal is generated when the limit value is exceeded.

37. The method of claim 26, wherein an impedance of at least one of the layers of the layer system is measured by impedance spectroscopy and a phase angle and/or an amount of the impedance is determined.

38. The method of claim 37, wherein different layers of a layer sequence are measured and phase angle displacements and/or differences in amount are determined.

39. The method of claim 38, wherein a selection of resonances typical of molecules with respect to aggregate states of analyzed layers is carried out by impedance spectroscopy.

40. The method of claim 38, wherein a concentration determined on the basis of the radiation of the degradation products and the phase angle displacements and/or differences in amount determined by impedance spectroscopy measurements are analyzed for congruence with respect to one another, and an indication signal is generated when no congruence is determined.

41. The method of claim 37, wherein either concentrations or impedances or concentrations and impedances are determined repeatedly over a period of time.

42. The method of claim 26, wherein at least one data connection to a virtual storage and computing unit is produced and steps of the method are carried out by the virtual storage and computing unit.

43. The method of claim 26, wherein method-related data are conveyed to a reception device of a recipient.

44. The method of claim 26, wherein detection is carried out on packaged foodstuff, the foodstuff being a layer of the layer system.

45. An apparatus for the detection of degradation products of biological molecules in layers of a layer system, wherein the apparatus comprises an excitation source for providing and emitting an excitation radiation, a detection unit for detecting radiation induced by excited degradation products, beam paths of the excitation source and of the detection unit being directed into a common measuring space, and a computing unit for controlling the excitation source and the detection unit and for evaluating detected radiation, wherein the excitation source is arranged above a layer system located in the measuring space, the layer system comprising at least a first through layer which is transparent to the excitation radiation and to radiation induced by excited degradation products, and at least a further layer after the through layer, and a beam path of the excitation source being directed under a radiation angle to the through layer in order to excite degradation products in at least one layer located below the through layer, and wherein the detection unit is arranged above the through layer in such a way that the radiation of the degradation products which is induced by the excitation radiation can be detected by the detection unit.

46. The apparatus of claim 45, wherein the beam paths of the excitation source and of the detection unit intersect in that layer in which the excitation of the degradation products takes place.

47. The apparatus of claim 45, wherein the excitation source is formed of a plurality of controllable excitation sources, each with its own beam paths.

48. A portable measuring device for mobile detection by the method of claim 26, wherein the measuring device comprises a receiving shell for receiving a telecommunications device.

49. The portable measuring device of claim 48, wherein an adapter by which a power supply of the measuring device is produced through the power source of the telecommunications device is arranged in the receiving shell, and it is possible to control the measuring device via a program which can be operated by the telecommunications device.

50. The portable measuring device of claim 49, wherein a data connection to a virtual storage and computing unit can be produced by the telecommunications device.
